(19) United States
(12) Patent Application Publication
(10) Pub. No.: US 2024/0374329 A1
(43) Pub. Date: Nov. 14, 2024

(54) ROBOTIC SYSTEM WITH FORCE MONITORING FOR COMPUTER-ASSISTED SURGERY SYSTEM

(71) Applicant: ORTHOSOFT ULC, Montreal (CA)

(72) Inventor: Louis-Philippe AMIOT, Montreal (CA)

(21) Appl. No.: 18/660,753

(22) Filed: May 10, 2024

Related U.S. Application Data
(60) Provisional application No. 63/501,755, filed on May 12, 2023.

(51) Int. Cl. A61B 34/37; A61B 34/10; A61B 34/20; A61B 90/00

(52) U.S. Cl. CPC A61B 34/37; A61B 34/10; A61B 2034/2046; A61B 2090/064

(57) ABSTRACT

A system for monitoring a force of an end effector of a robot on a bone in computer-assisted surgery, may have computer-readable program instructions executable by a processing unit for: obtaining tool tracking data for an end effector of the robot arm in a frame of reference of a bone; and continuously tracking and outputting the position and orientation of the end effector in the frame of reference, using the tool tracking data, and concurrently obtaining force sensor data pertaining to at least one force being applied by the robot arm on the bone.

### CROSS-REFERENCE TO RELATED APPLICATION

The present application claims the priority of U.S. Patent Application No. 63/501,755, filed on May 12, 2023 and incorporated herein in its entirety by reference.

### TECHNICAL FIELD

The present application relates to robotized computer-assisted surgery including bone and tool tracking, and robotically assisted tool manipulation.

### BACKGROUND OF THE ART

Tracking of surgical instruments or tools is an integral part of computer-assisted surgery (hereinafter “CAS”), including robotized CAS. The end effector, the tools, bodily parts are tracked for position and/or orientation in such a way that relative navigation information pertaining to bodily parts is obtained. The information is then used in various interventions (e.g., orthopedic surgery, neurological surgery) with respect to the body, such as bone alterations, implant positioning, incisions and the like during surgery.

In robotized CAS, tools may be supported by a robotic arm. By this support, the positioning of the tool relative to the body may be stabilized by the robotic assistance, removing the need for human muscle support. Moreover, the robotic arm may have encoders and like joint tracking to provide additional tracking data. Thus, while surgeons may have developed an expertise in manipulations performed during surgery, some practitioners prefer robotized assistance.

Surgeons perform many aspects of a surgical procedure basing themselves on experience. To produce systematic results, there is a need to monitor certain aspects of orthopedic procedures, gather data and analyze through various methods to continually refine surgical techniques.

There is also a need to deliver certain surgical steps under controlled conditions (such as force applied). This cannot be reliably done without a measurement device.

### SUMMARY

In accordance with a first aspect of the present disclosure, there is provided a system for monitoring a force of an end effector of a robot on a bone in computer-assisted surgery, comprising: a processing unit; and a non-transitory computer-readable memory communicatively coupled to the processing unit and comprising computer-readable program instructions executable by the processing unit for: obtaining tool tracking data for an end effector of the robot arm in a frame of reference of a bone; and continuously tracking the position and orientation of the end effector in the frame of reference, using the tool tracking data, and concurrently obtaining force sensor data pertaining to at least one force being applied by the end effector on the bone.

Further in accordance with the aspect, for instance, the computer-readable program instructions are executable by the processing unit for operating the robot arm in an autonomous mode as a function of the at least one force.

Still further in accordance with the aspect, for instance, operating the robot arm includes controlling the robot arm to increase or decrease the at least one force.

Still further in accordance with the aspect, for instance, operating the robot arm includes controlling the robot arm for the at least one force to remain within at least one force threshold.

Still further in accordance with the aspect, for instance, the computer-readable program instructions are executable by the processing unit for operating a tool at the end effector as a function of the at least one force.

Still further in accordance with the aspect, for instance, operating the tool at the end effector includes controlling the tool at the end effector to increase or decrease the at least one force.

Still further in accordance with the aspect, for instance, operating the robot arm includes controlling the robot arm for the at least one force to remain within at least one force threshold.

Still further in accordance with the aspect, for instance, operating the tool at the end effector includes controlling the tool at the end effector to increase or decrease a torque applied by the tool.

Still further in accordance with the aspect, for instance, operating the tool at the end effector includes stopping an operation of the tool at the end effector when a condition is met.

Still further in accordance with the aspect, for instance, stopping the operation of the tool at the end effector when the condition is met includes stopping the operation when a depth of penetration has been reached.

Still further in accordance with the aspect, for instance, stopping the operation of the tool at the end effector when the condition is met includes stopping the operation when a resistance against the tool is at a given level.

Still further in accordance with the aspect, for instance, the computer-readable program instructions are executable by the processing unit for operating a tool at the end effector according to a force profile.

Still further in accordance with the aspect, for instance, obtaining force sensor data includes calculating a resistance of the bone to the force applied by the end effector.

Still further in accordance with the aspect, for instance, continuously tracking the position and orientation of the end effector in the frame of reference includes calculating and outputting a depth of penetration of a tool of the end effector in the bone.

Still further in accordance with the aspect, for instance, the continuously tracking and the concurrently obtaining occur during a reaming action by a reamer tool on the bone.

Still further in accordance with the aspect, for instance, the computer-readable program instructions are executable by the processing unit for operating the robot arm in an collaborative mode as a function of the at least one force.

Still further in accordance with the aspect, for instance, operating the robot arm in the collaborative mode includes controlling the robot arm to support the end effector in a given position and orientation while an operator applies a force on the end effector and against the bone.

Still further in accordance with the aspect, for instance, operating the robot arm in the collaborative mode includes displaying the force sensor data in real time.

Still further in accordance with the aspect, for instance, the robot arm with at least one force sensor thereon is part of the system, the force sensor providing the force sensor data.

Still further in accordance with the aspect, for instance, an end effector supports a tool.

### DESCRIPTION OF THE DRAWINGS

FIG. 1 is a schematic view of a robotic system with force monitoring for computer assisted surgery in accordance with an aspect of the present disclosure, relative to a patient;

FIG. 2 is a block diagram of the robotized computer-assisted surgery of FIG. 1;

FIG. 3 is a schematic view of a robot arm joint sequence in accordance with an embodiment, with a reaming tool;

FIG. 4 is a schematic view of a robot arm joint sequence in accordance with another embodiment, with an impactor tool;

FIG. 5 is a computer-assisted method for force monitoring during a reaming action in a bone, in accordance with an aspect of the present disclosure; and

FIG. 6 is a series of graphs depicting exemplary force/torque profiles for operation of the robot arm.

### DETAILED DESCRIPTION

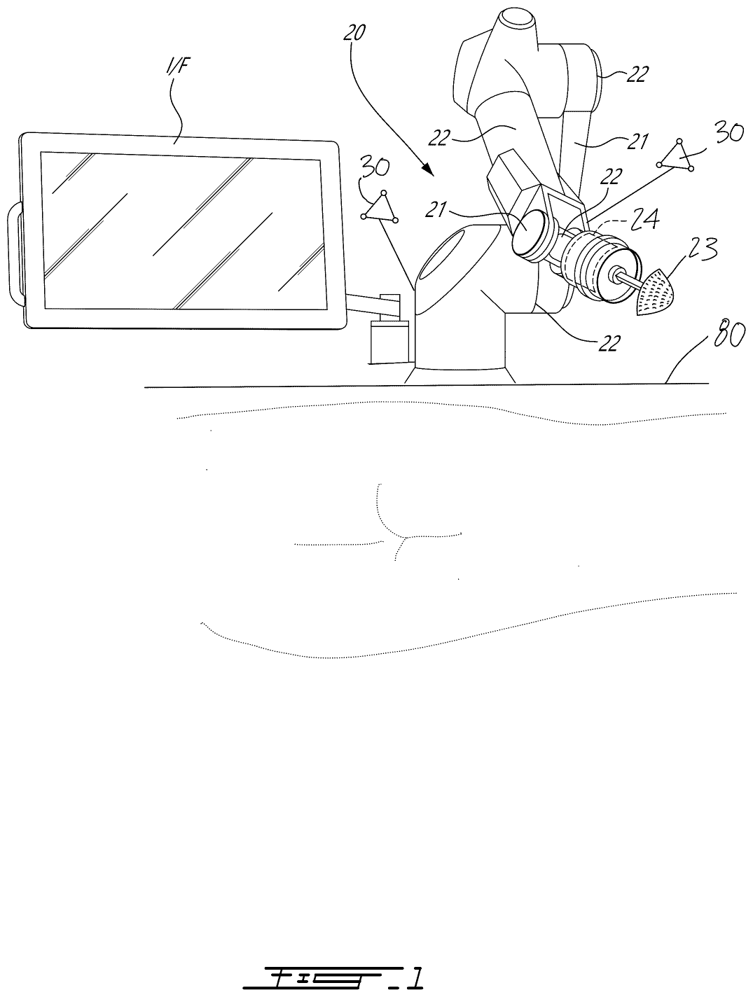

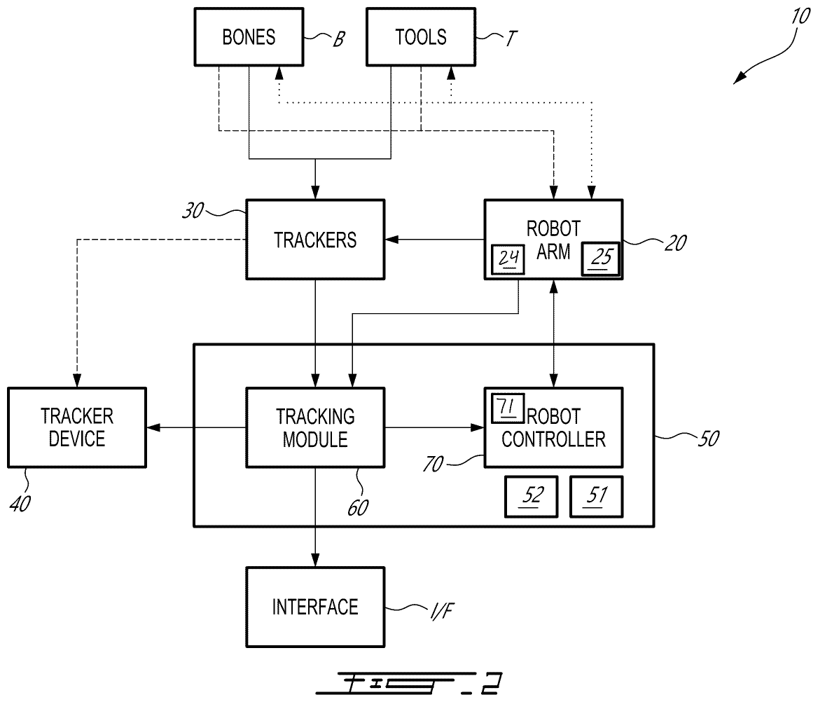

Referring to FIGS. 1 and 2, a robotic system with force monitoring for computer-assisted surgery (CAS) is generally shown at 10, and is used to provide surgery assistance to an operator. For simplicity, it will be referred to herein as the system 10. In FIG. 1, the system 10 is shown relative to a dummy patient in lateral decubitus, but only as an example. The system 10 could be used for any body parts, including non-exhaustively hip joint, spine, knee and shoulder bones, for orthopedic surgery, but could also be used in other types of surgery. For example, the system 10 could be used for surgery of all sorts, such as brain surgery, and soft tissue surgery.

The robotic system 10 may be robotized in a variant, and has, may have or may be used with a robot 20, optical trackers 30, a tracker device 40, a CAS controller 50 (also known as a super controller 50), a tracking module 60, and a robot controller 70 (also known as a robot driver), or any combination thereof:
The robot 20, shown by its robot arm, may be present as the working end of the system 10, and may be used to perform or guide bone alterations as planned by an operator and/or the CAS controller 50 and as controlled by the CAS controller 50. This may be referred to as an autonomous mode, as the robot 20 is not manipulated by a user. While a robot arm is displayed, the robot, robot system and robotic process may be implemented in the form of an orientation mechanism without a robotic arm. More specifically, interconnected links with joints that may be lockable supporting a tool may constitute a robot in accordance with the present disclosure. The robot arm 20 may also be configured for collaborative/cooperative mode in which the operator may manipulate the robot arm 20. For example, the tooling end, also known as end effector, may be manipulated by the operator while supported and oriented by the robot arm 20. The robot 20 may be the coordinate measuring machine (CMM) of the robotic system 10. In a variant, the robot 20 is mounted to the operating-room (OR) table 80, bed or like support platform on which the patient is laid, seated;
The optical trackers 30 are optionally positioned on the robot 20 (FIG. 2), on patient tissue (e.g., bones B), and/or on the tool(s) T and like surgical instruments, and provide tracking data for the robot 20, the patient and/or tools;
The tracking device 40, also known as a sensor device, apparatus, etc performs optical tracking of the optical trackers 30, so as to enable the tracking in space (a.k.a., navigation) of the robot 20, the patient and/or tools;
The optical trackers 30 and tracking device 40 are an optional tracking modality of the robotic system 10. Other camera(s) may be present, for instance as a complementary registration tool or as a primary tracking tool. The camera may for instance be mounted on the robot 20, such as on the robot arm, such that the point of view of the camera is known in the frame of reference, also known as the coordinate system. Tracking may also be done using the robot 20 and its joint tracking capacity, as an alternative, or in addition to the tracking modalities described above. Moreover, other tracking modalities may include the use of inertial sensors, etc;
The CAS controller 50, also known as the super controller, includes the processor(s) and appropriate hardware and software to run a computer-assisted surgery procedure in accordance with one or more workflows. The CAS controller 50 may include or operate the tracking device 40, the tracking module 60, and/or the robot controller 70. As described hereinafter, the CAS controller 50 may also drive the robot arm 20 through a planned surgical procedure;
The tracking module 60 is tasked with determining the position and/or orientation of the various relevant objects during the surgery procedure, such as the end effector of the robot arm 20, bone(s) B and tool(s) T, using data acquired by the tracking device 40 and by the robot 20, and/or obtained from the robot controller 70. The position and/or orientation may be used by the CAS controller 50 to control the robot arm 20;
The robot controller 70 is tasked with powering or controlling the various joints of the robot arm 20, based on operator demands or on surgery planning. The robot controller 70 may also optionally calculate robot movements of the robot arm 20, so as to control movements of the robot arm 20 autonomously in some instances, i.e., without intervention from the CAS controller 50.

Other components, devices, systems, may be present, such as surgical instruments and tools T, interfaces I/F such as displays, screens, computer station, servers, and like etc. Secondary tracking systems may also be used for redundancy.

Referring to FIG. 1, the robot 20 may have the robot arm 20 may be secured to the OR table 80 in a variant of the present disclosure. The robot arm 20 may alternatively stand from a base, for instance in a fixed relation relative to supporting the patient, whether it is attached to or detached from the OR table 80. The robot arm 20 has a plurality of joints 21 and links 22, of any appropriate form, to support an end effector 23 that may interface with the patient, or may be used during surgery without interfacing with the patient. In FIG. 1, merely as an example, the end effector 23 is shown as being a reamer, that is rotatably supported at the end of the robot arm 20 for rotating about its shaft axis. The end effector or tool head may optionally incorporate a force/torque sensor for collaborative/cooperative control mode, in which an operator manipulates the robot arm 20. The robot arm 20 is shown being a serial mechanism, arranged for the tool head 23 to be displaceable in a desired number of degrees of freedom (DOF). The tool head 23 may for example be a support that is not actuated, the support being used to support a tool, with the robot arm 20 used to position the tool relative to the patient. For example, the robot arm 20 controls 6-DOF movements of the tool head 23, i.e., X, Y, Z in the coordinate system, and pitch, roll and yaw. Fewer or additional DOFs may be present, as shown below with reference to FIG. 3. For simplicity, only a fragmented illustration of the joints 21 and links 22 is provided, but more joints 21 of different types may be present to move the end effector 23 in the manner described above. The joints 21 are powered for the robot arm 20 to move as controlled by the CAS controller 50 in the six DOFs, and in such a way that the position and orientation of the end effector 23 in the coordinate system may be known, for instance by readings from encoders on the various joints 21. Therefore, the powering of the joints is such that the end effector 23 of the robot arm 20 may execute precise movements, such as moving along a single direction in one translation DOF, or being restricted to moving along a plane, among possibilities. Such robot arms 20 are known, for instance as described in U.S. patent application Ser. No. 11/610,728, and incorporated herein by reference.

The end effector 23 of robot arm 20 may be defined by a chuck or like tool interface, typically actuatable in rotation. As a non-exhaustive example, numerous tools may be used as end effector for the robot arm 20, such tools including a registration pointer, a reamer (e.g., cylindrical, tapered), a reciprocating saw, a retractor, a camera, an ultrasound unit, a laser rangefinder or light-emitting device (e.g., the indicator device of U.S. Pat. No. 8,882,777), a laminar spreader, an instrument holder, or a cutting guide, depending on the nature of the surgery. The various tools may be part of a multi-mandible configuration or may be interchangeable, whether with human assistance, or as an automated process. The installation of a tool in the tool head may then require some calibration in order to track the installed tool in the X, Y, Z coordinate system of the robot arm 20.

The end effector 23 of the robot arm 20 may be positioned by the robot 20 relative to surgical area A in a desired orientation according to a surgical plan, such as a plan based on preoperative imaging. In order to position the end effector 23 of the robot arm 20 relative to the patient B, the CAS controller 50 can manipulate the robot arm 20 automatically (without human intervention), or by a surgeon manually operating the robot arm 20 (e.g. physically manipulating, via a remote controller through the interface I/F) to move the end effector 23 of the robot arm 20 to the desired location, e.g., a location called for by a surgical plan to align an instrument relative to the anatomy. Once aligned, a step of a surgical procedure can be performed, such as by using the end effector 23.

As shown in FIG. 2, the robot arm 20 may include a force sensor unit 24 in the end effector 23. The force sensor 24 may for example be a load cell, or other similar sensor, that is used to measure the force(s) applied to the body part (e.g., bone(s), soft tissue) by the end effector 23 (if the end effector 23 is a bone altering tool contacting the bone) or through the end effector 23 if the end effector 23 supports a bone-altering tool T, such as one triggered by a user. The force sensor 24 may bit a set of force sensors to measure different force vectors. The force sensor 24 may also include a torque sensor to measure the torque applied. The robot arm 20 may be configured to apply the force against the bone, and thus the force sensor unit 24 is used to measure the force(s) being applied, to allow a control of the robot arm 20 as a function of the force to be applied. This may include a combination of forces, such as the torque applied by a rotating tool (e.g., reamer) and the concurrent pressure applied onto the rotating tool by the robot arm 20, by the user applying pressure on the rotating tool supported by the robot arm 20, etc. The force sensor 24 (e.g., load cell) can detect activation of the end effector (for example start of the rotation of the reamer tool 23) and start applying a force progressively (or according to any force applying pattern) from then on. The amount and/or magnitude of force may have been chosen by the surgeon, over a period of time specified by the surgeon (e.g., force is increasing from zero to target in a given duration period, such as 1.5 seconds). Once rotation of the end effector has been stopped by the surgeon, the force may immediately return to zero. Force profiles are provided below with reference to FIG. 6.

The end effector such as the reamer tool 23 can also be configured to travel to a certain 3D target into the bone and stop automatically when the target has been reached, allowing accurate control of the penetration depth. This may be done via the tracking of the tool and bone.

The robot arm 20 may also include sensors 25 in its various joints 21 and links 22. The sensors 25 may be of any appropriate type, such as rotary encoders, optical sensors, position switches that are a non-exhaustive list of potential sensors, for the position and orientation of the end effector, and of the tool in the end effector 23 to be known. More particularly, the tracking module 60 may determine the position and orientation of the robot 20 in a frame of reference of the robot 20, such as by obtaining the position (x, y, z) and orientation (phi, theta, ro) of the end effector 23 from the CAS controller 50 using the sensors 25 in the robot arm 20, i.e., robot coordinates may be an integrated function of the robot 20 in that it may determine the position and orientation of its end effector 23 with respect to its coordinate system. Using the data from the sensors 25, the robot 20 may be the coordinate measuring machine (CMM) of the robotic system 10, with a frame of reference (e.g., coordinate system, referential system) of the procedure being relative to the fixed position of the base 20B of the robot 20. The sensors 25 must provide the precision and accuracy appropriate for surgical procedures. The coupling of tools to the robot arm 20 may automatically cause a registration of the position and orientation of the tools in the frame of reference of the robot 20, though steps of calibration could be performed.

Referring to FIG. 1, the trackers 30 are shown secured to the bones B and on the robot 20 (e.g., at one or more locations), and may also or alternatively be on instruments, such as the tools T. The trackers 30 may be known as trackable elements, markers, navigation markers, active sensors (e.g., wired or wireless) that may for example include infrared emitters. In a variant, the trackers 30 are passive retro-reflective elements, that reflect light. The trackers 30 have a known geometry so as to be recognizably through detection by the tracker device 40. For example, the trackers 30 may be retro-reflective lenses. Such trackers 30 may be hemispherical in shape, by way of a shield. The shield may be hollow and may cover a reflective membrane or surface. In an embodiment, the trackers 30 may be active emitters.

The tracker device 40 may be embodied by an image capture device, capable of illuminating its environment. In a variant, the tracker device 40 may have two (or more) points of view, such that triangulation can be used to determine the position of the tracker devices 30 in space, i.e., the coordinate system of the robotic system 10. The tracker device 40 may emit light, or use ambient light, to observe the trackers 30 from its points of view, so as to determine a position of the trackers 30 relative to itself. In an embodiment, the tracker device 40 is of the type known as the Polaris products by Northern Digital Inc. The tracker device 40 may form the complementary part of the CMM function of the robotic system 10, with the trackers 30 on the robot base 20 for example. The tracker device 40 may include a depth camera (e.g., Kinect) that has the capacity to track objects my its processing of its video feed. When the tracker device 40 is a depth camera, it may not need to be employed with trackers, or may rely on tokens or like flat visual markers.

Referring to FIG. 2, the CAS controller 50 is shown in greater detail relative to the other components of the robotic system 10. The CAS controller 50 has a processor unit 51 and a non-transitory computer-readable memory 52 communicatively coupled to the processing unit 51 and configured for executing computer-readable program instructions executable by the processing unit 51 to perform some functions, such as tracking the patient tissue and tools, using the position and orientation data from the robot 20 and the readings from the tracker device 40. Accordingly, as part of the operation of the CAS controller 50, the computer-readable program instructions may include an operating system that may be viewed by a user or operator as a GUI on one or more of the interfaces of the robotic system 10. It is via this or these interfaces that the user or operator may interface with the robotic system, be guided by a surgical workflow, obtain navigation data, etc. The CAS controller 50 may also control the movement of the robot arm 20 via the robot controller module 70. The robotic system 10 may comprise various types of interfaces I/F, for the information to be provided to the operator. The interfaces I/F may include and/or screens including wireless portable devices (e.g., phones, tablets), audio guidance, LED displays, head-mounted display for virtual reality, augmented reality, mixed reality, among many other possibilities. For example, the interface I/F comprises a graphic-user interface (GUI) operated by the system 10. The CAS controller 50 may also display images captured pre-operatively, or using cameras associated with the procedure (e.g., 3D camera, laparoscopic cameras, tool mounted cameras), for instance to be used in the collaborative/cooperative control mode of the system 10, or for visual supervision by the operator of the system 10, with augmented reality for example. The CAS controller 50 may drive the robot arm 20, in performing the surgical procedure based on the surgery planning achieved pre-operatively, or in maintaining a given position and orientation to support a tool. The CAS controller 50 may run various modules, in the form of algorithms, code, non-transient executable instructions, etc, in order to operate the robotic system 10 in the manner described herein. The CAS controller 50 may be part of any suitable processor unit, such as a personal computer or computers including laptops and desktops, tablets, server, etc.

The tracking module 60 may be a subpart of the CAS controller 50, or an independent module or system. The tracking module 60 receives the position and orientation data from the robot 20 and the readings from the tracker device 40. The tracking module 60 may hence determine the relative position of the objects relative to the robot arm 20 in a manner described below. The tracking module 60 may also be provided with models of the objects to be tracked. For example, the tracking module 60 may track bones and tools, and hence may use virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual tool models may be provided by the tool manufacturer, or may also be generated in any appropriate way so as to be a virtual 3D representation of the tool(s).

Additional data may also be available, such as tool orientation (e.g., axis data and geometry). By having access to bone and tool models, the tracking module 60 may obtain additional information, such as the axes related to bones or tools.

Still referring to FIG. 2, the CAS controller 50 may have the robot controller 70 integrated therein. However, the robot controller 70 may be physically separated from the CAS controller 50, for instance by being integrated into the robot 20 (e.g., in the robot base 20B). The robot controller 70 is tasked with powering and/or controlling the various joints of the robot arm 20. The robot controller 70 may also optionally calculate robot movements of the robot arm 20, so as to control movements of the robot arm 20 autonomously in some instances, i.e., without intervention from the CAS controller 50. There may be some force feedback provided by the robot arm 20, for instance via the force sensor 24 or other sensors 25 to avoid damaging the bones, to avoid impacting other parts of the patient or equipment and/or personnel. The robot controller 70 may perform actions based on a surgery planning. The surgery planning may be a module programmed specifically for any given patient, according to the parameters of surgery desired by an operator such as an engineer and/or surgeon. The parameters may include geometry of selected, planned bone cuts, planned cut depths, sequence or workflow of alterations with a sequence of surgical steps and tools, tools used, etc.

The robot controller 70 may include a force monitoring module 71. The force monitoring module 71 may be in the form of computer-readable program instructions to monitor the force applied by the robot arm 20 on the bone via the end effector 23, whether the end effector 23 is a support for a tool T, or a tool in and of itself. The force monitoring module 71 may then contribute to the driving actions of the robot controller 70. The force monitoring module 71 may receive sensor data from the force sensor unit 24, and optionally other data from other sensors, such as the sensors 25, and tracking data to contribute in determining the force vectors relative to the bone. Using this data, the force monitoring module 71 may determine if the force(s) being applied onto the bone is (are) within desired thresholds, whether the force is strictly provided by the human operator or by the robot arm 20, or a combination of forces in a collaborative mode. This may include the evaluation of force vectors, to ensure that an alteration trajectory is followed and/or that the forces are within thresholds based on the force vectors. Accordingly, the force monitoring module 71 obtains data from the tracking module 60 to track the force vectors as a function of the orientation of the body part. For example, in the case of hip surgery, once a reaming axis for a reamer of the acetabulum is selected, the end effector 23 with or without tool T is tracked, and force vectors are monitored relative to the reaming axis to ensure that the action of the end effector 23 is aligned with the reaming axis. The force monitoring module 71 may also include force profiles and/or force settings, or may apply a selected force, notably to avoid damaging bones or exceeding a depth of bone alterations. The force profiles may be in the form of functions, for example correlating the force applied to the speed of penetration, and such functions may be used by the force monitoring module 71 for the robot controller 70 to adjust its parameters, such as increasing/reducing applied forces, accelerating/decelerating tool speed, stopping and/or alerting the user. The force monitoring module 71 may also provide real-time force values, along with force thresholds, to allow human interventions.

As observed herein, the trackers 30 and the tracker device 40 may be complementary tracking technology. The position and orientation of the surgical tool calculated by the tracking module 60 using optical tracking may be redundant over the tracking data provided by the robot controller 70 and/or the CAS controller 50 and its embedded robot arm sensors 25. However, the redundancy may assist in ensuring the accuracy of the tracking of the surgical tool, and end effector 23. For example, the redundancy is used as a safeguard against incorrect tracking from the CAS controller 50, for instance due to relative movement between the robot 20, the tracker device 40, and the patient and/or table. Also, the tracking of the tool using the tracking module 60 may be used to detect any discrepancy between a calculated position and orientation of the surgical tool T through the sensors on the robot arm 20 and inertial sensor unit(s) 30, and the actual position and orientation of the surgical tool. For example, an improper mount of the tool T into the chuck of the robot arm 20 could be detected from the output of the tracking module 60, when verified by comparing the position and orientation from the CAS controller 50 (e.g., obtained from the encoders on the robot arm 20) with the optical tracking on the end effector 23. The operator may be prompted to verify the mount, via the interface I/F or head-mounted display 20. Moreover, the redundancy may enable the use of some of the trackers 30 as user interfaces, for the user to communicate with the CAS controller 50.

Consequently, the tracking module 60 may combine the optical tracking data from the tracker device 40 to the position and orientation data from the sensors 25 embedded in the robot arm 20, for the positional tracking data for the objects may be calculated by the tracking module 60, as detailed below. Therefore, the combination by the tracking module 60 of the tracking from the robot arm 20 and that from the tracker device 40 enable the tracking module 60 to track objects with a continuous and robust navigation data.

In an embodiment, the tracking module 60 uses a tracker 30 on the bone B or other body portion or OR table 80 to obtain the orientation of the bone B in the coordinate system, and locates the bone B using other methods, such as obtaining the position and orientation of a probing tool using the encoders in the robot arm 20, in a registration procedure described below. Stated differently, the bone B may be fixed on the OR table 80 and the system 10 may rely on trackers 30 fixed to the OR table 80 to optically track the bone B.

Referring to FIG. 3, an exemplary embodiment of the robot arm 20 is depicted, as being mounted to the OR table 80. The joints are shown as 21A, 21B, 21C etc, to facilitate a description thereof. The robot arm 20 may optionally be mounted to the OR table 80 by way of a prismatic joint 21A that may be locked. Accordingly, the robot arm 20 may be slid along the OR table 80 to be aligned with the patient. For example, the prismatic joint 21A may include a rail of the OR table 80. In a variant, there is no such prismatic joint 21A, with a link rigidly connected to the OR table 80 or other support, base, by which the robot arm 20 may be immobile relative to the OR table 80. It is also possible to have indexed discrete connection points for the robot arm 20 on the OR table 80 to allow a selection of a position of the robot arm 20 on a length of the OR table 80. Another prismatic joint 21B may optionally be present to allow a height adjustment for the robot arm 20. The prismatic joint 21B may also be a cylindrical joint. Joint 21B may be locked. Joint 21C allows one or two rotational degrees of freedom, to allow an orientation adjustment of the end effector 23, shown as being a reamer. The selection of the joint 21C may depend on whether prismatic joint 21B provides a rotational degrees of freedom. Thus, the robot arm 20 may have two or more rotational degrees of freedom, and one or more translational degree of freedom, two being shown in FIG. 3. The force sensor unit 24 is shown as being on a link that supports the reamer 23, but the force sensor unit 24 may also be on the end effector, and/or directly onto the bone altering tool. Accordingly, the force sensor unit 24 may track a force applied by the robot arm 20 on a bone, such as the pelvis. While FIG. 3 shows the reamer 23 integrated to the robot arm 20, the end effector of the robot arm 20 may be a support to which a reaming tool is attached (e.g., reamer, drill), such that the operator may trigger the reaming tool.

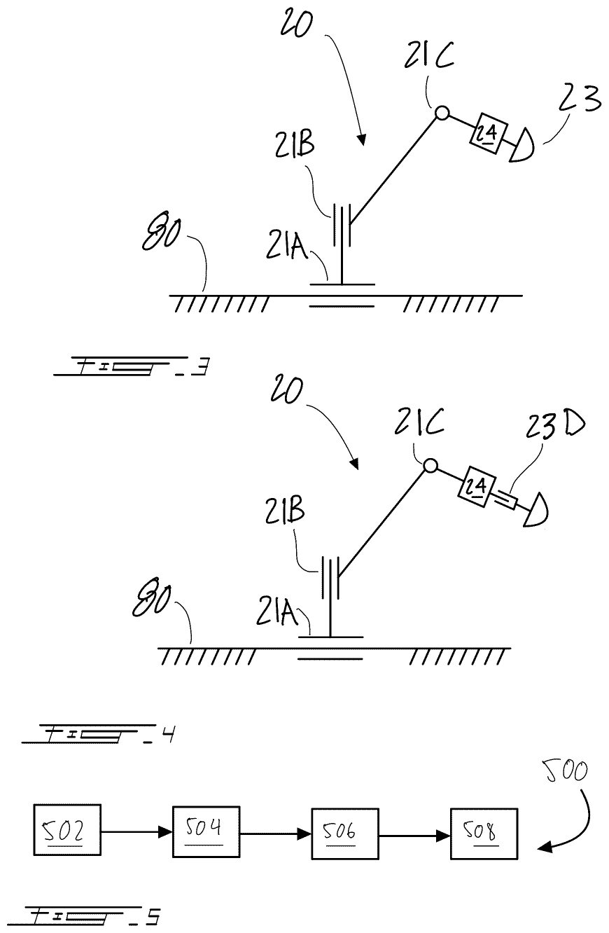

Referring to FIG. 4, another exemplary embodiment of the robot arm 20 is depicted, as being mounted to the OR table 80. The robot arm 20 bears similarities with that of the robot arm 20, but has an impaction or impactor tool at the end effector 23. The impactor tool 23 may a cylinder (e.g., pneumatic, hydraulic), a linear actuator (e.g., an electromechanical actuator as a ball-screw device), with a cup implant at its end. The joints are shown as 21A, 21B, 21C etc, to facilitate a description thereof. The robot arm 20 may optionally be mounted to the OR table 80 by way of a prismatic joint 21A that may be locked. Accordingly, the robot arm 20 may be slid along the OR table 80 to be aligned with the patient. For example, the prismatic joint 21A may include a rail of the OR table 80. In a variant, there is no such prismatic joint 21A, with a link rigidly connected to the OR table 80 or other support, base, by which the robot arm 20 may be immobile relative to the OR table 80. It is also possible to have indexed discrete connection points for the robot arm 20 on the OR table 80 to allow a selection of a position of the robot arm 20 on a length of the OR table 80. Another prismatic joint 21B may optionally be present to allow a height adjustment for the robot arm 20. The prismatic joint 21B may also be a cylindrical joint. Joint 21B may be locked. Joint 21C allows one or two rotational degrees of freedom, to allow an orientation adjustment of the end effector 23. The selection of the joint 21C may depend on whether prismatic joint 21B provides a rotational degrees of freedom. Thus, the robot arm 20 may have two or more rotational degrees of freedom, and one or more translational degree of freedom, two being shown in FIG. 4. The force sensor unit 24 is shown as being on a link that supports the impactor tool 23 but the force sensor unit 24 may also be on the end effector, and/or directly onto the impaction tool. Accordingly, the force sensor unit 24 may track a force applied by the robot arm 20 on the bone. While FIG. 4 shows the impactor tool 23 integrated to the robot arm 20, the end effector of the robot arm 20 may be a support to which the impactor tool is attached (e.g., drill).

In a variant, it is the surgeon or like personnel that operates the tool 23 supported by the robot 20. The robot 20 may be tasked with holding the tool 23 in a given orientation, i.e., along a given trajectory, and while applying a force based on force parameters set by the surgeon/personnel or by the system 10. The surgeon/personnel may pull the trigger of the tool 23 and apply a directional force that will be converted to the given force pattern by the robot 20.

Now that the various components of the robotic system 10 have been described, a contemplated procedure performed with the robotic system 10 or with a similar CAS system is set forth, with reference to FIGS. 1 and 2.

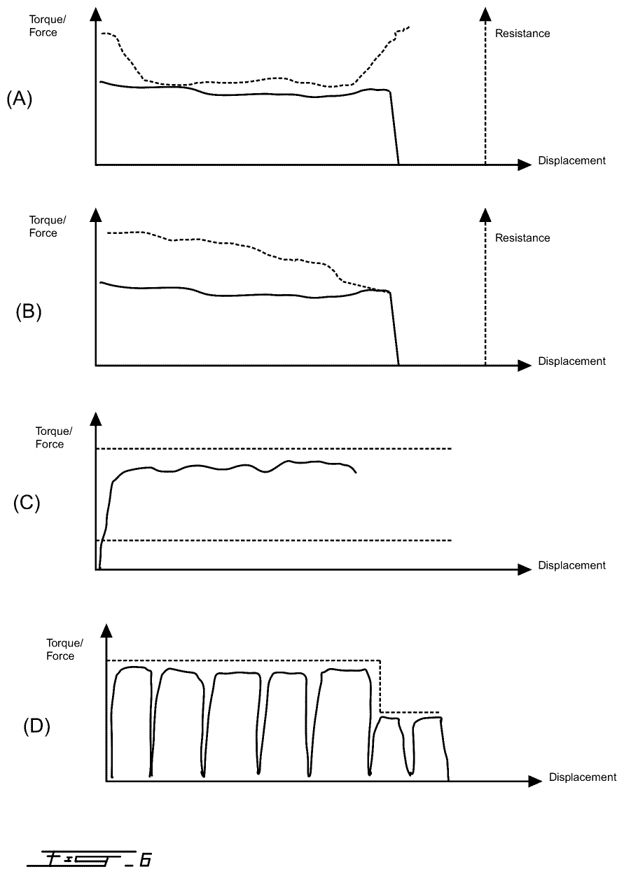

A flow chart illustrative of a method for tracking an end effector of a robot in computer-assisted surgery is shown at 500 in FIG. 5, and is an example of a procedure that may be performed by the CAS controller 50, robot controller 70 and/or other parts of the robotic system 10 of the present disclosure. For example, the method 500 may be computer-readable program instructions in the non-transitory computer-readable memory 52 for example, and executable by the processing unit 51 communicatively coupled to the processing unit 51.

According to step 502, tool tracking data (e.g., encoder data in the joints of the robot arm 20) may be obtained for an end effector 23 of the robot arm 20 in a frame of reference. The frame of reference is fixed in space and may include a position and orientation of a bone or other human body part to be altered, the position and orientation obtained in any appropriate way (e.g., digitizing of points, image processing, imaging (Xray, CT), etc). In an embodiment, the tool tracking data may be done using the sensors 25 in the robot arm 20. Step 502 may also include tracking an object, such as another robot arm 20, a tool used in free hand movement.

According to step 504, the position and orientation of the end effector 23 in the frame of reference is continuously tracked and output, using the tool tracking data, i.e., the robot coordinate tracking data. The CAS controller 50 may continuously output tracking data indicating the position and orientation of the end effector 23 in the frame of reference, for example relative to the object, body part, patient, also concurrently tracked in the frame of reference.

According to step 506, force sensor data is concurrently obtained with the continuous tracking of step 504. The force sensor data may pertain to one or more forces being applied by the robot arm on the bone, and may include force vector data. For example, the force sensor data may be obtained by the force sensor unit 24, on the robot arm 20 or in a tool supported by the robot arm 20. The force sensor data may include torque as well, and may include a resistance of the body part to the action of the tool supported by the robot arm 20.

According to step 508, the robot arm may be operated as a function of the at least one force. The operating may include operating the robot arm by controlling the robot arm to increase or decrease the at least one force; operating the robot arm includes controlling the robot arm for the at least one force to remain within at least one force threshold. The continuously tracking and the concurrently obtaining occur during a reaming action by a reamer tool on the bone. Operating the robot arm by controlling the robot arm to increase or decrease the at least one force may be as a function of a force profile, e.g., a force value as a function of depth of penetration, and/or speed of penetration. FIG. 6 provides different examples of force profiles. For example, with reference to (A) and (B) of FIG. 6, the magnitude of force may be reduced at or near completion of the alteration. In (A), the tool may for instance be a reciprocating saw, for which the force sensor(s) 24 can measure a resistance along a displacement of the saw (i.e., depth of penetration). The resistance may be greater as the distal cortical bone is reached, for instance when a tibia or femur is cut to define a plane. The robot arm 20 with tool T may be operated for the force applied to stop abruptly as the distal cortical bone may be an indication of a boundary of the bone. This may prevent or limit soft tissue damage.

Thus, force sensing may allow the system 10 to determine a variation in resistance of the body part (e.g., bone). This may indicate that the end effector 23/tool T may have reached a limit. For example, force sensing may indicate that the end effector 23/tool T has reached cancellous bone. In a variant shown in (B), as the end effector 23/tool T may gradually penetrate through the cortical bone, the force sensing (including torque sensing) may indicate to the system 10 that the increased rate of penetration, lower resistance encountered, that the cortical bone has been altered through. The system 10 may therefore automatically decelerate and/or stop or reduce the pressure applied by the robot arm 20.

Referring to FIG. 6, graph (C), force/torque thresholds are illustrated. The robot arm 20, tool T may be operated to remain within the force/torque thresholds. While an upper and a lower threshold are shown, a single threshold may be used as well, such as an upper threshold.

Referring to FIG. 6, graph (D), an exemplary force profile is shown for an impaction, such as the impaction of an acetabular cup using the arrangement of FIG. 4. In such a scenario, it may be desired to lessen the force of the impacts at or near full penetration of the acetabular cup. Therefore, as the cup is impacted, it may be desired that the last strokes of impaction be at a lower force.

The various profiles shown in FIG. 6 are examples among others. They illustrate that the actions of the tool T, robot arm 20 (whether autonomous or in collaborative mode) may be controlled for the forces to be applied in a predetermined and selected manner, mindful of the body part against which the force(s) are applied. The resistance of the body part may be derived from the force sensing, as the resistance may be indicative of what portion of the body part is recipient of the force. The system 10 may act in consequence, as cancellous bone, for example, may be exposed to lesser forces than cortical bone. While the X-axis of the various graphs shows a displacement or depth of penetration, it is considered to have profiles based on time, on speed of penetration.

The graphs of FIG. 6 may also be representative of a display provided to a user (e.g., surgeon and staff), whether the robot arm 20 is operated in collaborative mode or in autonomous mode, to guide the user as to the ongoing alteration. This may be done on the user interface I/F.

The robotic system 10 may perform continuous tracking. This means that the tracking may be performed continuously during at least discrete time periods of a surgical procedure. Continuous tracking may entail pauses, for example when the bone is not being altered. However, when tracking is required, the robotic system 10 may perform a continuous tracking output, with any disruption in the tracking output triggering an alarm or message to an operator. The methods described herein may limit or reduce disruptions in the tracking, notably due to movements of the robot 20 and/or tracker device 40. If movements are detected, the time required to recalibrate the robotic system 10.

In a variant, the present disclosure pertains to a system for monitoring a force of an end effector of a robot on a bone in computer-assisted surgery, that may include a processing unit; and a non-transitory computer-readable memory communicatively coupled to the processing unit and comprising computer-readable program instructions executable by the processing unit for: obtaining tool tracking data for an end effector of the robot arm in a frame of reference of a bone; and continuously tracking and outputting the position and orientation of the end effector in the frame of reference, using the tool tracking data, and concurrently obtaining force sensor data pertaining to at least one force being applied by the robot arm on the bone.

1. A system for monitoring a force of an end effector of a robot on a bone in computer-assisted surgery, comprising:
a processing unit; and
a non-transitory computer-readable memory communicatively coupled to the processing unit and comprising computer-readable program instructions executable by the processing unit for:
obtaining tool tracking data for an end effector of the robot arm in a frame of reference of a bone; and
continuously tracking the position and orientation of the end effector in the frame of reference, using the tool tracking data, and
concurrently obtaining force sensor data pertaining to at least one force being applied by the end effector on the bone.

2. The system according to claim 1, wherein the computer-readable program instructions are executable by the processing unit for operating the robot arm in an autonomous mode as a function of the at least one force.

3. The system according to claim 2, wherein operating the robot arm includes controlling the robot arm to increase or decrease the at least one force.

4. The system according to claim 2, wherein operating the robot arm includes controlling the robot arm for the at least one force to remain within at least one force threshold.

5. The system according to claim 1, wherein the computer-readable program instructions are executable by the processing unit for operating a tool at the end effector as a function of the at least one force.

6. The system according to claim 5, wherein operating the tool at the end effector includes controlling the tool at the end effector to increase or decrease the at least one force.

7. The system according to claim 6, wherein operating the robot arm includes controlling the robot arm for the at least one force to remain within at least one force threshold.

8. The system according to claim 6, wherein operating the tool at the end effector includes controlling the tool at the end effector to increase or decrease a torque applied by the tool.

9. The system according to claim 6, wherein operating the tool at the end effector includes stopping an operation of the tool at the end effector when a condition is met.

10. The system according to claim 9, wherein stopping the operation of the tool at the end effector when the condition is met includes stopping the operation when a depth of penetration has been reached.

11. The system according to claim 9, wherein stopping the operation of the tool at the end effector when the condition is met includes stopping the operation when a resistance against the tool is at a given level.

12. The system according to claim 1, wherein the computer-readable program instructions are executable by the processing unit for operating a tool at the end effector according to a force profile.

13. The system according to claim 1, wherein obtaining force sensor data includes calculating a resistance of the bone to the force applied by the end effector.

14. The system according to claim 1, wherein continuously tracking the position and orientation of the end effector in the frame of reference includes calculating and outputting a depth of penetration of a tool of the end effector in the bone.

15. The system according to claim 1, wherein the continuously tracking and the concurrently obtaining occur during a reaming action by a reamer tool on the bone.

16. The system according to claim 1, wherein the computer-readable program instructions are executable by the processing unit for operating the robot arm in an collaborative mode as a function of the at least one force.

17. The system according to claim 16, wherein operating the robot arm in the collaborative mode includes controlling the robot arm to support the end effector in a given position and orientation while an operator applies a force on the end effector and against the bone.

18. The system according to claim 17, wherein operating the robot arm in the collaborative mode includes displaying the force sensor data in real time.

19. The system according to claim 1, including the robot arm with at least one force sensor thereon, the force sensor providing the force sensor data.

20. The system according to claim 19, further including an end effector supporting a tool.
